(19) United States
(12) Patent Application Publication
(10) Pub. No.: US 2010/0209483 A1
(43) Pub. Date: Aug. 19, 2010

(54) TRANSDERMAL DELIVERY OF MEPTAZINOL

(76) Inventor: Richard Franklin, Fleet (GB)

Correspondence Address: FROMMER LAWRENCE & HAUG, 745 FIFTH AVENUE- 10TH FL., NEW YORK, NY, 10151, US

(21) Appl. No.: 12/377,287

(22) PCT Filed: Aug. 14, 2007
(86) PCT No.: PCT/US07/75902

§ 371 Date: Sep. 2, 2009

Related U.S. Application Data
(63) Continuation-in-part of application No. 11/614,165, filed on Dec. 21, 2006.
(60) Provisional application No. 60/822,318, filed on Aug. 14, 2006.
(60) Provisional application No. 60/862,114, filed on Oct. 19, 2006.
(60) Provisional application No. 60/862,114, filed on Oct. 19, 2006.
(60) Provisional application No. 60/753,357, filed on Dec. 21, 2005.

(51) Int. Cl. A61K 9/70; A61K 31/55; A61K 9/14; A61K 8/49; A61P 29/00; A61Q 17/04; A61Q 15/00; A61Q 1/02; A61Q 19/02

(52) U.S. Cl. 424/448; 424/449; 424/400; 514/212.01; 424/484; 424/59; 424/65; 424/63; 424/62

(57) ABSTRACT

A delivery system for the delivery of a salt of meptazinol which increases the bioavailability of meptanizol by an effective amount to provide analgesic relief is disclosed. One embodiment of the delivery system is a transdermal device which increases the skin flux of meptazinol by an effective amount to provide analgesic relief. Also disclosed are methods of providing analgesic relief.

### RELATED APPLICATIONS AND INCORPORATION BY REFERENCE

This application claims priority to U.S. Provisional Application No. 60/822,318 filed 14 Aug. 2006. Reference is also made to U.S. application Ser. No. 11/614,165 filed 21 Dec. 2006, International Application No. PCT/US2006/048783 filed 21 Dec. 2006, and U.S. Provisional Application No. 60/862,114 filed on 19 Oct. 2006, and U.S. Provisional Application No. 60/753,357 filed on 21 Dec. 2005.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This invention relates to the administration of meptazinol for analgesic purposes and more particularly to a method and device for administering meptazinol to a patient in need thereof over an extended period of time at an essentially constant rate while avoiding first pass metabolism.

### BACKGROUND OF THE INVENTION

Meptazinol is a mixed agonist-antagonist analgesic with specificity for the mμ1 opioid receptor and its chemical structure is defined by formula (I) below:

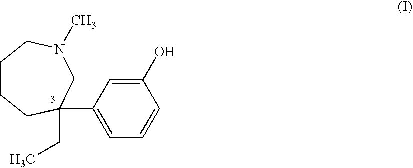

Preparation of meptazinol hydrobromide salt was referred to in U.S. Pat. No. 3,729,465 and preparation of the free base form of meptazinol was referred to in U.S. Pat. No. 4,197,241, both of which are incorporated by reference.

Meptazinol has been shown to have a negligible clinical dependency liability from both formal clinical investigation and the lack of reported instances of street use/abuse. The lack of addictive potential for meptazinol was first reported in 1987 by the internationally reknowned investigator, Dr. Don Jasinski (Lexington, Ky.). This property distinguishes meptazinol from many other strong analgesics such as fentanyl (e.g. Duragesic), pentazocine, oxycodone (e.g. Oxycontin, Percocet), and morphine which are all classified as “Controlled Drugs” with consequent prescription/dispensation restrictions.

Meptazinol also has many clinical advantages over the more conventional opioid analgesics which include causing minimal respiratory depression, causing minimal sedation and lacking a constipating effect.

Causing minimal respiratory depression makes meptazinol a favored obstetric analgesic to avoid infant respiratory distress. Other analgesics given during labor such as pethidine and diacetylmorphine can cause significant infant respiratory depression giving rise to the so-called grey baby syndrome often necessitating the use of a narcotic antagonist such as naloxone to reverse this effect.

Causing minimal sedation is advantageous in treating chronic pain conditions and assists a patient in conducting a normal daily life. The sedation associated with other analgesics frequently induces lethargy and a dramatic reduction in the quality of life—with the patients entering a near twilight world.

Lacking a constipating effect is an important property in treating chronic pain. The constipation commonly associated with the other strong analgesics can be a most distressing condition especially for the older patient. For this group of patients, frequently the target population for strong analgesics, the lack of a constipating effect for meptazinol represents an important advantage over other strong analgesics such as pethidine.

Additionally, age is unlikely to affect the clearance of meptazinol which is effected by a simple one-step glucuronidation process with the ensuing inactive, water-soluble conjugate being filtered at the kidney. This process of conjugative metabolic clearance is not as affected by age as some other clearance mechanisms such as direct filtration of the active entity at the kidney or oxidative metabolic clearance as required for example by pethidine.

However, despite these clinical advantages, use of meptazinol has been restricted by two major disadvantages: (1) low oral biovailability; with reported mean values lying between 4-9% as the result of extensive first pass metabolism and (2) a propensity, in common with other strong analgesics, to cause nausea and emesis. The nausea and emesis worsens bioavailability due to physical drug loss by vomiting. Furthermore, since meptazinol is known to inhibit gastric emptying and effectively traps part of the orally dosed drug in the stomach, greater quantities of meptazinol may be lost through such emesis. All these factors lead to highly variable plasma drug levels of meptazinol after oral dosing and consequently a variable patient response. Such is the demand for immediate relief from moderate to severe pain that a patient may be unwilling to continue treatment with meptazinol until an optimal dosage is discovered for their personal use. This frustration, in attaining optimal dosage levels for each individual patient, can lead to compliance problems and ineffective medication and pain relief. The compliance issue is further exacerbated by the need for frequent oral administration of meptazinol, typically 4-6 times per day as a result of its short plasma half-life (1.5-2.0 hours).

Transdermal delivery of strong analgesics in recent years has proven to be a useful means of overcoming many of the problems associated with their oral administration. Modulating the sharp rise in plasma drug levels, usually seen after oral dosing, may serve to minimize the emesis associated with the comparatively high Cₘₐₓ values resulting from rapid absorption. In the specific case of meptazinol, avoidance of emesis becomes very much more important to minimize loss of drug trapped in the stomach by its inhibitory effects on gastric emptying.

Transdermal delivery also provides a means of avoiding the first pass metabolism through the liver which in the case of meptazinol removes up to 98.1% of an oral dose. Such a high first pass elimination of the drug inevitably leads to large inter and intra subject variability in achieved plasma drug concentrations. For example, in one publication (Norbury H. M, Franklin, R. A, Graham, D. F., Eur. J. Clin. Pharm., vol. 25, pgs 77-80, (1983)) oral bioavailability varied from 1.89% to 18.5%, almost a ten-fold range.

Meptazinol is inherently not a potent drug when administered orally, requiring 200 mg dosages every four to six hours. Even when the poor bioavailability of meptazinol is factored in, the average daily required dose for an effective dose would be ˜50-100 mg which approximates to a flux rate of ˜83-166 ug/cm²/h from a 25 cm² transdermal patch. Such inherently high flux rates are not usually seen with other transdermal products and so represents a significant technical challenge.

Examples of transdermal delivery systems which may generically incorporate meptazinol have been referred to in the art, e.g. Oshlack et al. (U.S. Pat. No. 6,716,449—hereinafter “Oschlack”), but there is no evidence in the art that these systems were capable of delivering meptazinol at the necessary high flux rates. Oschlack makes reference to maintaining prior art defined concentration levels or release rates while decreasing the amount of dosing by a factor of about 100 to about 1000, i.e. Oschlack does not indicate how the flux rate of meptazinol could be achieved by transdermal delivery.

Skin flux can be determined by multiplying the permeability coefficient (kₚ in cm/h) of meptazinol by the aqueous solubility of meptazinol. The aqueous solubility of meptazinol (free base) is 0.17 mg/mL and the permeability coefficient of meptazinol can be calculated using the empirical formula:

log kₚ=−2.7+0.71 log P−0.0061 MW (MW for meptazinol is 233.35)

This results in an estimated skin flux of just 5.6 μg/cm²/h for meptazinol which is roughly 15-30× lower than the flux rate necessary to achieve analgesic effects through transdermal delivery.

Therefore, a need still exists in the art for a transdermal delivery system for a non-addictive mixed agonist-antagonist analgesic such as meptazinol to achieve a sufficiently high flux rate to deliver a pharmacologically effective amount of the drug to treat pain or provide analgesic relief.

Citation or identification of any document in this application is not an admission that such document is available as prior art to the present invention.

### SUMMARY OF THE INVENTION

Surprisingly, the applicants have found that the disadvantages in the art with respect to the use of meptazinol can be overcome by the combination of various ternary and quaternary delivery vehicles in a transdermal device, (and most unexpectedly) with use of particular salt forms of meptazinol to provide sufficiently high flux rates to achieve plasma concentrations effective for analgesic relief.

Thus, it is an object of this invention to provide a delivery system which avoids first pass metabolism and delivers a pharmacologically effective amount of meptazinol for pain or to provide analgesic relief. The invention provides a viable means of avoiding the very large first pass effect seen with meptazinol after oral dosing. This invention will result in lower variability in achieved plasma concentrations, improved analgesic efficacy and better patient compliance.

Patient compliance will be further improved by the requirement for less frequent dosage due to the sustained plasma concentrations achieved from this transdermal delivery device.

Additionally, the relatively slower rise in plasma drug concentrations is expected to minimize the drug's emetic effects which again will contribute to minimizing variability in analgesically effective plasma drug concentrations and improving patient compliance

The terms “delivery system” and “delivery vehicle” as used herein is meant to describe a method of providing meptazinol via transdermal transportation which avoids “first pass metabolism”. First pass metabolism refers to the reduction of bioavailability of a drug, e.g. meptazinol, because of the metabolic or excretory capacity of the liver which is a common problem associated with oral administration. Transdermal delivery is distinct from parenteral or delivery by injection in that the latter bypasses the stratum corneum, epidermal and dermal layers of the skin and delivers the active agent directly to the subcutaneous layer. Transdermal delivery as used herein is meant to describe a process wherein an active agent, e.g. meptazinol or a derivative thereof, contacts and passes through or permeates through one or more of the stratum corneum, epidermal and dermal layers of the skin. This passing through or permeation through can be accomplished via:
(1) transcellular penetration (across the cells);
(2) intercellular penetration (between the cells); or
(3) transappendageal penetration (via hair follicles, sweat and sebum glands, and pilosebaceous apparatus).

The invention disclosed herein is meant to encompass all pharmaceutically acceptable salts thereof for meptazinol (including those of the weakly acidic phenolic function as well as those of the weakly basic azepine nitrogen). Furthermore, it encompasses various other meptazinol precursors derived by covalent linkage to the phenolic function such as ethers esters and glycosides described later. The pharmaceutically acceptable salts (of the phenol) include, but are not limited to, metal salts such as sodium salt, potassium salt, cesium salt and the like; alkaline earth metals such as calcium salt, magnesium salt and the like; organic amine salts such as triethylamine guanidine & N-substituted guanidine salts, acetamidine & N-substituted acetamidine salts, pyridine salt, picoline salt, ethanolamine salt, triethanolamine salt, dicyclohexylamine salt, N,N′-dibenzylethylenediamine salt and the like. Pharmaceutically acceptable salts (of the azepine) include, but are not limited to inorganic acid salts such as hydrochloride, hydrobromide, sulfate, phosphate and the like; organic acid salts such as trifluoroacetate, maleate, and the like; sulfonates such as methanesulfonate, ethanesulphonate, benzenesulfonate, p-toluenesulfonate, camphor sulphonate and naphthalenesulphonate, and the like; amino acid salts such as alaninate, asparginate, glutamate and the like.

Meptazinol is a chiral molecule containing one stereogenic center at the C-3 position of the azepine and can therefore exist as two enantiomeric forms (R and S stereoisomers) Reference to meptazinol for the purposes of this invention encompasses each enantiomer and mixtures thereof including a racemic mixture (racemate) of the enantiomers unless otherwise indicated.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are apparent from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 illustrates the comparative permeation of meptazinol free base and a number of its salts through human skin.

FIG. 2 shows the skin flux of the various salts of meptazinol though human skin.

### DETAILED DESCRIPTION

The present invention is directed to a delivery system which delivers a pharmacologically effective amount of meptazinol for pain or to provide analgesic relief. Examples of other such delivery systems, include but are not limited to those means which enable delivery of meptazinol or salt form thereof via parenteral injection, pulmonary absorption, topical application, sublingual administration and rectal administration. Parenteral injections include delivery via intravenous injection, subcutaneous injection, intramuscular injection, intraarterial injection and intrathecal injection. Pulmonary absorption includes the use of inhalants and aerosols. Topical administration includes administration via: (1) mucous membranes which includes but is not limited to mucous membranes of the conjunctiva, nasopharnyx, oropharynx, vagina, colon, urethra and urinary bladder; (2) the skin (which includes topical or transdermal delivery); and (3) the eye.

In one embodiment of the invention, the delivery vehicle is for topical administration to the skin and includes but is not limited to a transdermal device, a cream, a lotion or an ointment which delivers a pharmacologically effective amount of meptazinol for pain or to provide analgesic relief. In another embodiment of the invention the delivery vehicle is a transdermal device.

The transdermal device is intended to deliver the pharmacologically effective amount of meptazinol either in a manner which: (1) controls the rate of drug delivery to the skin or (2) allows the skin to control the rate of drug absorption.

The transdermal device for the transdermal delivery of an effective amount of meptazinol to provide analgesic relief comprises of:
(a) a device which comprises of
   (i) a backing layer;
   (ii) a reservoir layer for the salt form of meptazinol or a salt of a meptazinol precursor;
   (iii) optionally a control membrane or non controlling microporous membrane;
   (iv) optionally an adhesive; and
   (v) optionally a protective peel strip; and
(b) a salt form of meptazinol or salt of a meptazinol precursor in an amount which results in delivery of an effective amount of meptazinol when added into the device and said device is applied to the skin; and
(c) a pharmaceutically effective carrier.

The backing layer, reservoir layer, control membrane, adhesive and protective peel strip can be formed using conventional teachings in the art such as those referred to in U.S. Pat. No. 6,818,226 (Dermal penetration enhancers and drug delivery systems involving same); U.S. Pat. No. 6,791,003 (Dual adhesive transdermal drug delivery system); U.S. Pat. No. 6,787,149 (Topical application of opioid analgesic drugs such as morphine); U.S. Pat. No. 6,716,449 (Controlled release compositions containing opioid agonist and antagonist); U.S. Pat. No. 5,858,393 (Transdermal formulation); U.S. Pat. No. 5,612,382 (Composition for percutaneous absorption of pharmaceutically active ingredients); U.S. Pat. No. 5,464,387 (Transdermal delivery device); U.S. Pat. No. 5,023,085 (Transdermal flux enhancers in combination with iontophoresis in topical administration of pharmaceuticals; U.S. Pat. No. 4,891,377 (Trandermal delivery of the narcotic analgesics etorphine and analogs); U.S. Pat. No. 4,654,209 (Preparation of percutaneous administration), each of which is incorporated by reference.

In another embodiment of the invention, the delivery device for topical administration is a transvaginal ring such as those described in U.S. Pat. Nos. 6,503,190; 6,394,094; 5,972,372; 5,694,947; 5,543,150; 3,920,805 and U.S. Patent Application Publications 2005-042292; 2003-152625 and 2002-090390. Generally, a vaginal ring has a body dimensioned to allow for insertion into the vagina, e.g. a cylindrical shape although other shapes can also be used, and can be configured by those of ordinary skill in the art to deliver the meptazinol salts of the invention. Delivery of the meptazinol in this manner may effect pain relief by virtue of the drug's historically reported local anaesthetic activity—about 1/10^{th} of that of lidocaine—or by a centrally mediated mechanism. Such an application could, for example, find application in the period immediately following gynaecological surgery.

In a preferred embodiment of the invention, the non-controlling membrane comprises Solupor 10P05A (manufactured by DSM) and the adhesive DURO-TAK 87-608A (manufactured by National Starch) in which the drug is dissolved in a vehicle comprising oleic acid, dimethyl isosorbide, propylene glycol and ethanol (in a ration of 3:2:70:25, respectively)

Alternatively, the transdermal device may constitute a so-called “drug in adhesive” or matrix patch in which the drug is intimately distributed in an appropriate pressure sensitive adhesive such as but not limited to the DURO-TAK polyacrylates. The transdermal device of the invention is able to provide long lasting relief and is an improvement from the prior art which require 4-6 dosages per day.

In one embodiment of the invention, the transdermal device is able to provide up to about 8 hours of analgesic relief; in another embodiment of the invention, the transdermal device is able to provide about 8 to about 24 hours of relief; and in a further embodiment of the invention, the transdermal device is able to provide from about 24 hours of relief to about 168 hours of relief.

Given the low solubility of the free base form of meptazinol free base (0.17 mg/mL in aqueous solution), it may be advantageous to derivatize the meptazinol to form a precursor compound which will degrade into meptazinol when traversing the layer(s) of the skin. Therefore, another embodiment of the invention is the delivery of meptazinol transdermally which is achieved by a transdermal device which contains a precursor of meptazinol which includes but is not limited to meptazinol esters, glycosides, salts of meptazinol or mixtures thereof. Precursors of meptazinol are compounds which undergo a transformation in vivo to produce meptanizol (e.g. cleavage of an ester bond, glycolysis, formation of the free base from the salt). Meptazinol esters, ethers and glycosides of the invention are compounds of the formula (II):

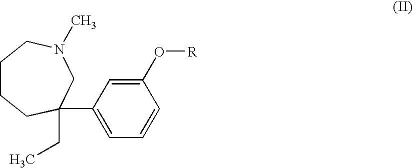

wherein R is an acyl group, a mono-, oligo- or poly-saccharide, or salts of mono-, oligo- or poly-saccharides. (Oligosaccharide for the purpose of this invention indicates a saccharide comprised of 2-10 monosaccharide units which are covalently bonded together)

When R forms an ester, one embodiment of the invention is where R is a —C(═O)—C₁-C₁₂-alkyl; yet another embodiment is where R is —C(═O)—C₁-C₁₂-alkyl-NR₁R₂ wherein R₁ and R₂ are independently hydrogen or C₁-C₄ alkyl; yet another embodiment is where R is C(═O)—C₁-C₁₂-alkylCO₂R₃ wherein R₃ is hydrogen, C₁-C₄ alkyl or is a cation.

In a further embodiment of the invention, R is a —C(═O)—C₁-C₄-alkyl; yet another embodiment is where R is —C(═O)—C₁-C₄-alkyl-NR₁R₂ wherein R₁ and R₂ are independently hydrogen or C₁-C₄ alkyl; yet another embodiment is where R is C(═O)—C₁-C₄-alkylCO₂R₃ wherein R₃ is hydrogen, C₁-C₄ alkyl or is a cation.

When R forms an ether, one embodiment of the invention is where R is a substituted or unsubstituted C₁-C₁₂-alkyl or substituted or unsubstituted aryl. In another embodiment of when R is an ether, R is a substituted or unsubstituted C₁-C₄-alkyl or substituted or unsubstituted phenyl. In both embodiments, the substituents are selected from the group consisted of halogen, C₁-C₄-alkyl, and C₁-C₄-alkoxy.

When R is a monosaccharide, one embodiment of the invention is where R is selected from the group consisting of erythrosyl, threosyl, ribosyl, arabinosyl, xylosyl, lyxosyl, allosyl, altrosyl, glucosyl, glucosylamino, mannosyl, gulosyl, idosyl, galactosyl, galactosylamino, talosyl and salts thereof; another embodiment is where R is glucosyl, glucosylamino, galactosyl or galactosylamino and salts thereof; and yet another embodiment of the invention is where R is glucosyl and salts thereof.

When R is an oligosaccharide, one embodiment of the invention is where R is selected from the group consisting of lactose, sucrose, trehalose, Lewis a trisaccharide, 3′-O-sulfonato Lewis a, Lewis b tetrasaccharide, Lewis x trisaccharide, Sialyl Lewis x, 3′-O-sulfonato Lewis x, Lewis y tetrasaccharide and salts thereof.

When R is a polysaccharide, one embodiment of the invention is where R is selected from the group consisting of chitin, chitosan, cyclodextrin, dextran and pullulan; another embodiment of the invention is where the cyclodextrin is α-, β- or γ-cyclodextrin; yet another embodiment of the invention is where the cyclodextrin is β-cyclodextrin, dimethyl-β-cyclodextrin or hydroxypropyl-β-cyclodextrin.

As the cyclodextrin have a cavity which can accommodate the inclusion of a compound such as meptazinol, another embodiment of the invention is where the cyclodextrins described in R above can also be added to meptazinol to form an inclusion complex rather than being linked covalently.

In another embodiment of the invention, the meptazinol precusor is a salt and R is hydrogen but absent, whereby the oxygen is negatively charged; one embodiment of the invention is where the salt form is selected from the group consisting of sodium, potassium, caesium, calcium, magnesium, guanidine & N-substituted guanidine salts and acetamidine & N-substituted acetamidine salts triethylamine, pyridine, picoline, ethanolamine, triethanolamine, dicyclohexylamine, N,N′-dibenzylethylenediamine. Another embodiment of the invention is when R is hydrogen—or one of the aforementioned substituents—and the azepine nitrogen is positively charged and linked with hydrochloride, hydrobromide, sulfate, phosphate, formate, acetate, trifluoroacetate, maleate, tartrate, methanesulfonate, ethanesulphonate, benzenesulfonate, p-toluenesulfonate, naphthalene sulphonate, camphor sulfonate, arginate, alaninate, asparginate, glutamate and mixtures thereof.

Surprisingly, and contrary to prior notions that lower melting points (mp) are normally associated with improved skin permeability, the azepine salts of meptazinol do not show such a relationship. For example, meptazinol hydrochloride (mp 184° C.) was a much better permeant than the maleate (mp 102-104° C.). The hydrochloride also displayed a higher flux rate than the camsylate (mp of 46-48° C.).

Furthermore, and again in contrast to prior notions in the art, additional unexpected results occurred when using salts of meptazinol for transdermal delivery. Usually the free base is the preferred form of a drug for transdermal delivery due to its greater lipophilicity. For example, the skin flux of fentanyl free base is up to five times faster than the salt form. However, for meptazinol, the free base shows unexpectedly poor flux in comparison to the various salt forms. For example, meptazinol hydrochloride has a substantially greater flux than the free base. Previous reports in the scientific literature have suggested that ion pairs, i.e. salts, may improve transdermal flux by virtue of beneficially enhancing the physicochemical characteristics of the molecule. Such strategies have often employed lipophilic counter ions. Surprisingly, in the case of meptazinol, the use of more lipophilic counter ions such as the camsylate and tosylate were less effective in improving flux than the use of salts of stronger acid such as trifluoroacetic acid or hydrochloric acid.

In another embodiment of the invention, an additional analgesic can be added to the transdermal device. Examples of analgesics include but are not limited to ethanol, non-steroidal anti-inflammatory drugs (NSAIDs) and other compounds with analgesic properties such as but not limited to amitriptyline and carbamazepine.

In another embodiment of the invention, the pharmaceutically effective carrier includes but is not limited to a solvent such as alcohol, isopropylmyristate, glycerol monooleate or a diol such as propylene glycol, or the like. The delivery of the meptazinol or meptazinol precursor is enhanced by the use of a permeation enhancer which may also be included in the pharmaceutically effective carrier. In one embodiment of the invention, suitable permeation enhancers include but are not limited to polyunsaturated fatty acids (PUFA) such as arachidonic acid, lauric acid, α-linolenic acid, linoleic acid and oleic acid; dimethylisosorbide; azones; cyclopentadecalactone; alkyl-2-(N,N-disubstituted amino)-alkanoate ester (NexAct); 2-(n-nonyl)-1,3-dioxaolane (SEPA); cod-liver oil; essential oils, glycerol monoethers derived from saturated fatty alcohols; D-limonene; menthol and menthol ethyl ether; N-methyl-2-pyrrolidone (NMP); phospholipids; squalene; terpenes; and alcohols such as methanol, ethanol, propanol and butanol. see e.g. Pharmaceutical Skin Penetration Enhancement, ed. Walters et al., Marcel Dekker, Inc., (1993); Williams et al., “Penetration Enhancers”, Adv. Drug Deliv. Rev., vol. 56, pgs 603-618, (2004).

In another embodiment of the invention, transdermal drug delivery is enhanced by iontophoresis, magnetophoresis, or sonophoresis. Iontophoresis involves the delivery of charged chemical compounds across the skin membrane using an applied electrical field. see e.g. “Pharmaceutical Dosage Forms and Drug Delivery Systems—Chapter 10—Transdermal Drug Delivery Systems, Ointments, Creams, Lotions and Other Preparations”, ed. by Ansel et al., Williams & Wilkins, page 360, (1995). Magnetophoresis involves the use of a magnetic field to enhance drug delivery to the skin. see e.g. Murthy et al., “Physical and Chemical Permeation Enhancers in Transdermal Delivery of Terbutaline Sulphate”, AAPS PharmSciTech. 2001; 2(1). Sonophoresis is the use of high-frequency ultrasound which serves to compromise the integrity of the stratum corneum layer and improve permeability of compounds through the skin.

Alternatively, in another embodiment of the invention transdermal drug delivery may be effected using various topically applied ointments, creams, or lotions. Typically these may comprise oil-in-water emulsions or water-in-oil emulsions incorporating meptazinol or meptazinol precursor in one of the preferred vehicles. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents

In another embodiment of the invention, the solubility (as measured in aqueous solution) of the meptazinol or meptazinol precursor is about 30 mg/mL to about 500 mg/mL; in yet another embodiment of the invention, the solubility is about 50 mg/mL to about 400 mg/mL; and in a still further embodiment of the invention, the solubility is about 75 mg/mL to about 300 mg/mL.

In another embodiment of the invention, the skin flux for the delivery of the meptazinol or meptazinol precursor is about 20 to about 1000 μg/cm²/h; in yet another embodiment of the invention, the skin flux for the delivery of the meptazinol or meptazinol precursor is about 50 to about 500 μg/cm²/h; and in a further embodiment of the invention is about 75 to about 250 μg/cm²/h. In another embodiment of the invention the pH of the environment into which the meptazinol or meptazinol precursor is released is about pH 4.0 to about pH 7.0; in another embodiment, the pH is about 4.0 to about 6.0; and in a further embodiment, the pH is about 4.0 to about 5.0.

Optionally, additional skin care ingredients may be combined with meptazinol or the meptazinol precursors for their art recognized effects, these include abrasives, absorbents, adhesives, antiacne agents, anticaking agents, anticareis agents, antidandruff agents, antifoaming agents, antifungal agents, antimicrobial agents, antioxidants, antiperspirant agents, antistatic agents, binders, buffering agents, bulking agents, chelating agents, colorants, corn/callus/wart removers, corrosion inhibitors, cosmetic astringents, cosmetic biocides, denaturants, depilating agents, drug astringents, emollients, emulsion stabilizers, epilating agents, exfoliants, external analgesics, film formers, flavoring agents, fragrance ingredients, humectants, lytic agents, occlusives opacifying agents, oxidizing agents, pesticides, pH adjusters, plasticizers, preservatives, propellants, reducing agents, skin-bleaching agents, skin-conditioning agents, skin protectants, slip modifiers, solvents, sunscreen agents, surface modifiers, surfactants (including cleansing agents, emulsifying agents, foam boosters, hydrotopes, solubilizing agents, suspending agents), suspending agents (non-surfactant), ultraviolet light absorbers, viscosity controlling agents, viscosity decreasing agents, viscosity increasing agents (aqueous), viscosity increasing agents (non-aqueous) and mixtures thereof.

In another embodiment of the invention, use of the transdermal device described hereinabove can be used to provide analgesic effects to treat systemic or localized pain to a patient in need thereof.

Other advantages and characteristics of the invention will become apparent on reading the following description, given by way of non-limiting examples.

### Examples

### Example 1

### Improved Skin Flux by Using Meptazinol HCl Salt

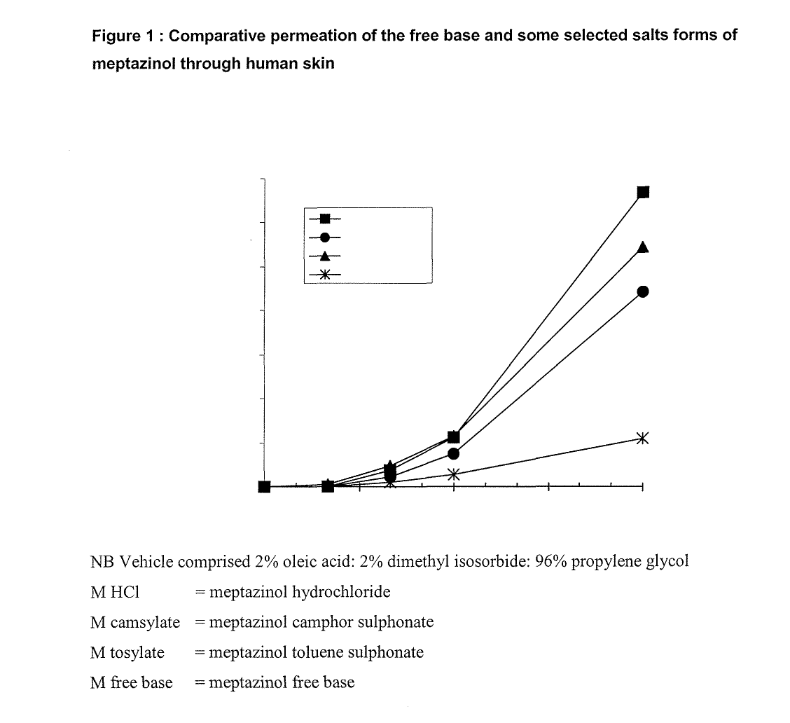

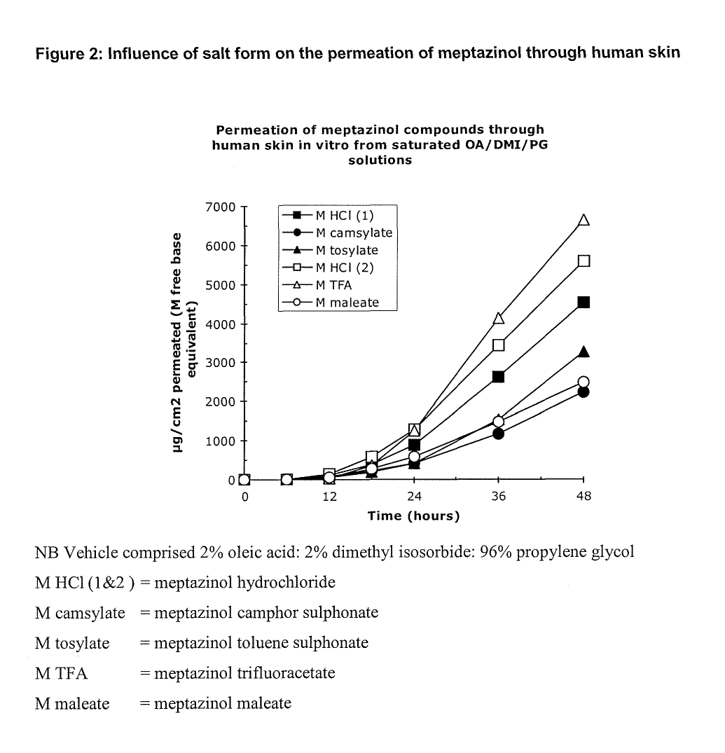

Using human skin in a conventional Franz cell in vitro apparatus the transdermal permeation of meptazinol was measured by assaying the amount of drug in the receptor fluid beneath the skin sample at various times after application to the skin. FIG. 1 shows that a salt of meptazinol is surprisingly more permeable than the free base form of meptazinol. FIG. 2 shows that surprisingly meptazinol salts formed from a stronger acid, such as the hydrochloride and trifluoroacetate salt are more rapidly absorbed than are those of weaker organic acids such as the camsylate, tosylate or maleate.

The data presented in Table 1 below show that under the test conditions cited above, the mean flux for the various salts tested were suitable for producing concentrations of meptazinol sufficient to produce a long-lasting effect when administered to patient in need thereof.

TABLE 1
Intersubject variability in flux rates for meptazinol salts through
human skin

| | | | Flux | |
|---|---|---|---|---|
| Applied | Cell | Donor | ug/ | Mean |
| compound | ID | number | cm²/h | flux ± sd |
| M HCl (1) | 1 | 281 | 197.1 | 173.3 ± 35.2 |
| M HCl (1) | 2 | 282 | 194.0 | |
| M HCl (1) | 3 | 284 | 162.5 | |
| M HCl (1) | 4 | 287 | 135.7 | |
| M HCl (1) | 5 | 289 | 218.0 | |
| M HCl (1) | 6 | 295 | 132.6 | |
| M camsylate | 7 | 281 | 78.1 | 79.2 ± 71.2 |
| M camsylate | 8 | 282 | 110.9 | |
| M camsylate | 9 | 284 | 27.3 | |
| M camsylate | 10 | 287 | 45.1 | |
| M camsylate | 11 | 289 | 204.2 | |
| M camsylate | 12 | 295 | 9.5 | |
| M tosylate | 13 | 281 | 158.8 | 135.9 ± 82.5 |
| M tosylate | 14 | 282 | 165.0 | |
| M tosylate | 15 | 284 | 90.9 | |
| M tosylate | 16 | 287 | 39.6 | |
| M tosylate | 17 | 289 | 273.7 | |
| M tosylate | 18 | 295 | 87.4 | |
| M HCl (2) | 19 | 292 | 66.5 | 179.0 ± 75.1 |
| M HCl (2) | 20 | 288 | 222.6 | |
| M HCl (2) | 21 | 282 | 213.4 | |
| M HCl (2) | 22 | 281 | 213.4 | |
| M TFA | 23 | 292 | 29.1 | 224.4 ± 167.6 |
| M TFA | 24 | 288 | 422.2 | |
| M TFA | 25 | 282 | 163.8 | |
| M TFA | 26 | 281 | 282.3 | |
| M maleate | 27 | 292 | 41.3 | 78.0 ± 24.7 |
| M maleate | 28 | 288 | 91.3 | |
| M maleate | 29 | 282 | 85.3 | |
| M maleate | 30 | 281 | 93.9 | |

NB Vehicle comprised 2% oleic acid: 2% dimethyl isosorbide: 96% propylene glycol

The data in Table 2 was obtained using the same in vitro Franz cell technique verifies that there was surprisingly no correlation between lower melting points and higher solubilities with overall skin flux rates. For example, based on prior notions in the art, meptazinol hydrochloride which has a higher melting point than meptazinol free base, would have been expected to have a worse skin flux rate but instead is several times better than the meptazinol free base. Likewise, meptazinol hydrochloride which is the salt of a stronger acid, has both lower solubility and higher melting point than meptazinol camsylate, tosylate or maleate which is the salt of a weaker acid, and yet still have an unexpectedly better skin flux rate.

TABLE 2
Saturated solubilities (& melting points) of meptazinol free base
and selected salts in a potential delivery vehicle comprising 2% oleic
acid: 2% dimethyl isosorbide: 96% propylene glycol

| Compound | Solubility at 32° C. (mg/ml) | MP (° C.) |
|---|---|---|
| Meptazinol hydrochloride | 78* | 184-186 |
| Meptazinol camsylate | ~250** | 46-48 |
| Meptazinol tosylate | ~140** | 40-42 |
| Meptazinol trifluoroacetate | 170* | 112-114 |
| Meptazinol maleate | 105* | 102-104 |
| Meptazinol free base | ~20** | 128-133 |

*Measured by HPLC
**Estimated from visual assessment only

Having thus described in detail various embodiments of the present invention, it is to be understood that the invention defined by the above paragraphs is not to be limited to particular details set forth in the above description as many apparent variations thereof are possible without departing from the spirit or scope of the present invention.

What is claimed is:

1. A transdermal device for the transdermal delivery of an effective amount of meptazinol to provide analgesic relief which comprises of:
(a) a device which comprises of
(i) a backing layer;
(ii) a reservoir layer for a salt of meptazinol or a salt of a meptazinol precursor;
(iii) optionally a control membrane or microporous non rate controlling membrane;
(iv) optionally an adhesive; and
(v) optionally a protective peel strip; and
(b) a salt of a meptazinol a salt of a meptazinol precursor in an amount which results in delivery of an effective amount of meptazinol when added into the device and said device is applied to the skin; and
(c) a pharmaceutically effective carrier.

2. The transdermal device of claim 1, wherein the device further comprises a control membrane and an adhesive and optionally a protective peel strip.

3. The transdermal device of claim 1, wherein the device is able to provide analgesic relief for a time period selected from the group consisting of about 8 hours of analgesic relief; and about 24 hours of relief to about 168 hours of relief.

4. The transdermal device of claim 1, which comprises a salt of a meptazinol precursor.

5. The transdermal device of claim 4, wherein the meptazinol precursor has the formula (II): 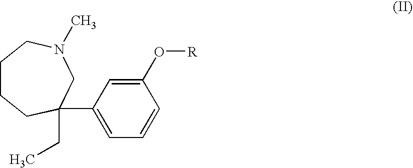
wherein,
R forms an ester, ether or glycoside with —O—; or
wherein
R is hydrogen or absent, whereby the oxygen is negatively charged, a salt of meptazinol.

6. The transdermal device of claim 5, wherein:
when R forms an ester, R is a —C(═O)—C₁-C₁₂-alkyl; —C(═O)—C₁-C₁₂-alkyl-NR₁R₂ wherein R₁ and R₂ are independently hydrogen or C₁-C₄ alkyl; or R is C(═O)—C₁-C₁₂-alkylCO₂R₃ wherein R₃ is hydrogen, C₁-C₄ alkyl or is a cation;
when R forms an ether, R is a substituted or unsubstituted C₁-C₁₂-alkyl or substituted or unsubstituted aryl;
when R forms a glycoside, R is selected from the group consisting of monosaccharide, oligosaccharide, polysaccharide, erythrosyl, threosyl, ribosyl, arabinosyl, xylosyl, lyxosyl, allosyl, altrosyl, glucosyl, glucosylamino, mannosyl, gulosyl, idosyl, galactosyl, galactosylamino, talosyl and salts thereof; another embodiment is where R is glucosyl, glucosylamino, galactosyl, galactosylamino, lactose, sucrose, trehalose, Lewis a trisaccharide, 3′-O-sulfonato Lewis a, Lewis b tetrasaccharide, Lewis x trisaccharide, Sialyl Lewis x, 3′-O-sulfonato Lewis x, Lewis y tetrasaccharide, chitin, chitosan, cyclodextrin, dextran, pullulan; α-cyclodextrin, β-cyclodextrin, γ-cyclodextrin, dimethyl-β-cyclodextrin, hydroxypropyl-β-cyclodextrin and salts thereof; or where R is an amino acid e.g. —C-alkyl NH₂.
when R is hydrogen but absent, and the oxygen is negatively charged, a salt of meptazinol wherein the salt form is selected from the group consisting of sodium, potassium, secium, calcium, magnesium, triethylamine, pyridine, picoline, ethanolamine, triethanolamine, dicyclohexylamine, N,N′-dibenzylethylenediamine.

7. The transdermal device of claim 6, wherein R is hydrogen and the nitrogen in formula (II) is positively charged and the salt form is hydrochloride, hydrobromide, sulfate, phosphate, trifluoroacetate, maleate, tartrate, methanesulfonate, ethanesulphonate, benzenesulfonate, p-toluenesulfonate, naphthalene sulphonate, camphor sulfonate, alaninate, asparginate, glutamate or mixtures thereof.

8. The transdermal device of claim 7, wherein the salt form is hydrochloride, camphor sulphonate, toluene sulfonate, trifluoroacetate, maleate or mixtures thereof.

9. The transdermal device of claim 8, wherein the salt of meptazinol has a solubility selected from the group consisting of about 30 mg/mL to about 500 mg/mL; about 50 mg/mL to about 400 mg/mL; and about 75 mg/mL to about 300 mg/mL.

10. The transdermal device of claim 8, wherein the salt of meptazinol has a skin flux selected from the group consisting of about 20 to about 1000 μg/cm²/h; about 50 to about 500 μg/cm²/h; and about 75 to about 250 μg/cm²/h.

11. The transdermal device of claim 8, wherein the salt of meptazinol is released into an skin environment with a pH selected from the group consisting of about pH 4.0 to about pH 7.0; about pH 4.0 to about pH 6.0; and about pH 4.0 to about pH 5.0.

12. The transdermal device of claim 1, which comprises of a salt of meptazinol wherein the salt form is hydrochloride, trifluoroacetate or mixtures thereof, has a solubility of about 75 mg/mL to about 300 mg/mL and a skin flux of about 75 to about 250 μg/cm²/h.

13. The transdermal device of claim 10, which additionally comprises abrasives, absorbents, adhesives, antiacne agents, anticaking agents, anticareis agents, antidandruff agents, antifoaming agents, antifungal agents, antimicrobial agents, antioxidants, antiperspirant agents, antistatic agents, binders, buffering agents, bulking agents, chelating agents, colorants, corn/callus/wart removers, corrosion inhibitors, cosmetic astringents, cosmetic biocides, denaturants, depilating agents, drug astringents, emollients, emulsion stabilizers, epilating agents, exfoliants, external analgesics, film formers, flavoring agents, fragrance ingredients, humectants, lytic agents, occlusives, opacifying agents, oxidizing agents, pesticides, pH adjusters, plasticizers, preservatives, propellants, reducing agents, skin-bleaching agents, skin-conditioning agents, skin protectants, slip modifiers, solvents, sunscreen agents, surface modifiers, surfactants (including cleansing agents, emulsifying agents, foam boosters, hydrotopes, solubilizing agents, suspending agents), suspending agents (non-surfactant), ultraviolet light absorbers, viscosity controlling agents, viscosity decreasing agents, viscosity increasing agents (aqueous), viscosity increasing agents (non-aqueous) and mixtures thereof.

14. The transdermal device of claim 12, which additionally comprises abrasives, absorbents, adhesives, antiacne agents, anticaking agents, anticareis agents, antidandruff agents, antifoaming agents, antifungal agents, antimicrobial agents, antioxidants, antiperspirant agents, antistatic agents, binders, buffering agents, bulking agents, chelating agents, colorants, corn/callus/wart removers, corrosion inhibitors, cosmetic astringents, cosmetic biocides, denaturants, depilating agents, drug astringents, emollients, emulsion stabilizers, epilating agents, exfoliants, external analgesics, film formers, flavoring agents, fragrance ingredients, humectants, lytic agents, occlusives, opacifying agents, oxidizing agents, pesticides, pH adjusters, plasticizers, preservatives, propellants, reducing agents, skin-bleaching agents, skin-conditioning agents, skin protectants, slip modifiers, solvents, sunscreen agents, surface modifiers, surfactants (including cleansing agents, emulsifying agents, foam boosters, hydrotopes, solubilizing agents, suspending agents), suspending agents (non-surfactant), ultraviolet light absorbers, viscosity controlling agents, viscosity decreasing agents, viscosity increasing agents (aqueous), viscosity increasing agents (non-aqueous) and mixtures thereof.

15. A method of providing analgesic effect to a patient in need thereof which comprises administering the transdermal device of claim 1.

16. The method of claim 15, wherein the analgesic effect is localized.

17. The method of claim 15, wherein the analgesic effect is systemic.

18. The method of claim 15, wherein administration of the transdermal device is accompanied by iontophoresis.

19. The method of claim 15, wherein administration of the transdermal device is accompanied by magnetophoresis.

20. The method of claim 15, wherein administration of the transdermal device is accompanied by sonophoresis.

21. The method of claim 15, wherein transdermal delivery is effected by use of a lotion, cream or ointment in place of a device.

22. The method of claim 15, wherein transdermal delivery is effected using a matrix patch in which the drug is dissolved in a suitable pressure sensitive adhesive.
